(19) United States
(12) Patent Application Publication
(10) Pub. No.: US 2013/0095477 A1
(43) Pub. Date: Apr. 18, 2013

(54) Non-Invasive Method for the Early Detection of Stomach Cancer

(71) Applicant: Alejandro Corvalán, Santiago (CL)

(72) Inventor: Alejandro Corvalán, Santiago (CL)

(21) Appl. No.: 13/498,750

(22) PCT Filed: Sep. 27, 2010
(86) PCT No.: PCT/CL10/00041

§ 371 Date: Jan. 4, 2013

(30) Foreign Application Priority Data
Sep. 28, 2009 (CL) 1907-2009

(51) Int. Cl. C12Q 1/68

(52) U.S. Cl. CPC C12Q 1/6886
USPC 435/6.11; 435/6.14

(57) ABSTRACT

The invention relates to a non-invasive method for the early detection of stomach cancer, based on the identification of a biomarker. The biomarker corresponds to the methylated promoter region of the Reprimo gene.

### STATE OF THE ART

The gastric cancer is the fourth most common cancer and the second leading cause of cancer related deaths in the world (1). Despite the improvements in its treatment, the prognosi is negative because invasive stages are frequently detected (2). When the disease is confined to the mucouse or submucouse layers of the stomach (early stages), the 5 year survival rate is 95%. By contrast, when extended to the muscularis propia or serosa layers (advanced stages), the 5 year survival rate drops to 10-20% (3).

The diagnosis of gastric cancer in the early stages is difficult because the majority of them are asymptomatic until advanced stage (3).

In recent years, the pathogenesis of cancer had been attributed to particular genetic alterations in genes related to the genesis of tumors (i.e., gene amplification and point mutations) (4). However, these alterations vary according to histological subtypes, indicating distinct carcinogenetic pathways of gastric cancer (5). Presently, the focus of the study of cancer has been oriented to epigenetic alterations. The epigenetic processes control the regulating sequences of the genes through hypermethylation and the packaging and functionality of the human genome through acetylation, thus contributing to the normal development and to varied diseases (6). The epigenetic field opens a new vision of the pathogenesis of cancer because the quantitative detection potential of these regulatory modifications of the genes (7).

The first epigenetic alteration documented in gastric cancer was the hypermethylation of the DNA mismatch repair genes (hMSH2 and hMLH1, reference 8). Subsequently, several other specific tumor suppressor genes have been described as inactivated by hypermethylation (5). However, and although recent reports explore gene methylation of specific genes (9-11) a comprehensive DNA methylation profile in gastric cancer has not been carried out.

Because of the necessity of having methods for the early diagnosis, to prevent the high mortality rate of advanced gastric cancer, it is necessary to search for new methods for the early diagnosis that is suitable for application to an asymptomatic population, in a fast, low cost, and non invasive form.

Because of this, the inventors focused in studying biomarkers that can be used in the gastric cancer detection methods.

Up to now, biomarkers in non invasive samples that directly detect gastric cancer in asymptomatic populations had not been disclosed. The best known are the gastric atrophy markers, a lesion occurring before the appearance of gastric cancer. This lesion has a 5-10% risk of developing gastric cancer, implying that these patients shall require special surveillance using invasive methods (radiology and endoscopy).

On the other hand, gastric atrophy, apart from being a gastric cancer precursor lesion is a lesion associated to aging. Therefore, its predicting value is lost with the age increase of the population undergoing the evaluation.

The results obtained in present invention provide additional information on the significance of the epigenetic modification in gastric cancer and of the methylation profiles of the histological variants of gastric cancer.

Thus, the results obtained by present invention are relevant from the clinical perspective, in that they consider that the hypermethylation of the Reprimo gene promoter is a potential candidate for the early detection of gastric cancer.

### DISCLOSURE OF THE INVENTION

Present knowledge indicates that the prognosis of gastric cancer correlates to the prognosis of tumoral invasion (2, 3). A two time's reduction of mortality caused by gastric cancer was achieved by prospective studies with photofluorographic methods (relative risk=0.52; confidence interval at 95%=0.36-0.74) among the individuals undergoing and not undergoing the test (53). However, the massive application of this type of program of analysis in the asymptomatic population represents a high cost.

On the other hand, an alternative non invasive method for the massive detection of gastric cancer is the “serologic biopsy” comprising detection of pepsinogen and gastrine 17, for the detection of gastric atrophy that is a precursory lesion of gastric cancer (54). However, as it was mentioned above, this is a lesion associated to aging, and not directly associated to cancer.

The prior alternatives lead to the necessity for developing new methodologies for the early detection of gastric cancer.

In present invention, a non invasive method using the Reprimo gene in the early detection of gastric cancer is disclosed, because it distinguishes in non invasive samples (plasma), asymptomatic individuals from patients with gastric cancer.

The method disclosed by present invention may be applied in massive detection programs, thus reducing the high cost of traditional invasive methods (radiology and endoscopy).

The early detection method of present invention is based on the finding of genes with aberrant hypermethylation, and based on the selection of candidate genes by the search of CpG islands in promoter regions of 24 genes, in 32 retrospective cases of advanced gastric cancer. Table 1 summarizes the name, location, function of the genes and the reference to the methodology for each one of the 24 genes selected in this study. The determination of the methylation status of the 24 selected genes was made by methylation-specific PCR (16). The selected genes are tumor repressor genes. The CpG methylation in these sites is associated with the silencing of genes comprising six essential alterations in the physiology of malignant cells that collectively dictate malignant growth (self-sufficiency in growth signals, insensitivity to growth-inhibitory signals, evasion of programmed cell death, limitless replicative potential, sustained angiogenesis, and tissue invasion and metastasis), and they had previously been described as undergoing hypermethylation in other tumor types (16-33)

TABLE 1
summary of data of the genes tested for aberrant hypermethylation on gastric cancer promoters

| Gene | | | | |
|---|---|---|---|---|
| (abbreviation) | Name of the gene | Location | Function | Reference |
| 3OST | Heparan sulfate (glucosamine) | 16p12 | Angiogenesis | 15 |
| APC | Adenomatus polyposis coli gene | 5q21 | Signal transduction | 16 |
| BLU | Homologous to the MTG/ETO | 3p21.3 | Cell cycle regulation | 14 |
| | family of transcription factors | | | |
| BRCA1 | Breast cancer 1 gene | 17q2 | DNA repair | 17 |
| COX2 | Cyclooxygenase 2 | 1q25 | Angiogenesis | 18 |
| DAPK | Death-associated protein kinase | 9q34 | Evasion of programmed | 19 |
| | | | cell death | |
| CDH1 | E-cadherin | 16q22 | Tissue invasion and | 19 |
| | | | metastasis | |
| ER | Estrogen Receptor | 6q25.1 | DNA binding, activation, | 20 |
| | | | transcription | |
| FHIT | Fragile histidine triad | 3p14.2 | Evasion of programmed | 14 |
| | | | cell death | |
| GSTp1 | Homologous to the MTG/ETO | 3p21.3 | Cell cycle regulation | 21 |
| | family of transcription factors | | | |
| hMLH1 | Human homologues of the MutL | 3p21.3 | DNA repair (Mismatch | 22 |
| | genes of bacteria | | repair genes) | |
| MGMT | O-6 methylguanine-DNA | 10q26 | DNA repair | 23 |
| | methyltransferase | | | |
| p14 | Cyclin-dependent kinase | 9p21 | Cell cycle regulation | 16 |
| | inhibitor 2B | | | |
| p15 | Cyclin-dependent kinase | 9p21 | Cell cycle regulation | 24 |
| | inhibitor 2B | | | |
| p16 | Cyclin-dependent kinase | 9p21 | Cell cycle regulation | 16 |
| | inhibitor 2A | | | |
| p73 | Cyclin-dependent kinase | 9p21 | Angiogenesis | 19 |
| | inhibitor 2A | | | |
| PTEN | Phosphatase and tensin | 10q23 | Signal transduction | 25 |
| | homologue | | | |
| RAR-β | Retinoic acid receptor β 2 gene | 3p24 | DNA binding, activation | 26 |
| | | | transcription | |
| Reprimo | TT53 dependent G₂ arrest | 2q23 | Cell cycle regulation | 27 |
| | mediator candidate | | | |
| RIZI | Rb-Interacting zinc finger gene 1 | 1p36 | Methyltransferase | 19 |
| | | | superfamily | |
| RUNX3 | Runt-related transcription factor | 1p36 | Signal transduction | 28 |
| | 3 | | (TGF-β pathway) | |
| SEMA3B | Semaphorin 3B | 3p21.3 | Evasion of programmed | 29 |
| | | | cell death | |
| SHP1 | Hematopoietic cell-specific | 12p13 | Signal Transduction | 30 |
| | protein-tyrosine phosphatase SH | | (JAK-STAT pathway) | |
| | PTP1 | | | |
| TIMP3 | Tissue Inhibitor of | 22q12 | Tissue invasion and | 31 |
| | metalloproteinase-3 | | metastasis | |

A detailed description of the invention follows.

Description of the Technique Employed.

Clinical Samples

For identifying the DNA methylation patterns of the 24 genes, evaluation-validation tests were employed (12). The evaluation group corresponds to 32 cases of gastrectomies retrospectively collected with gastric cancer diagnosis according to the World Health Organization (WHO) (13). This group corresponds to 21 men (65.6%) with an age average of 65 years old, 11 of these tumors (34.3%) were located in the cardia, 7 (21.8%) in the middle third, and 14 (43.7%) located in the antrum. Four tumors (12.5%) at early stage and 22 tumors (68.8%) were lymph node positive. Thirteen tumors (40.6%) were signet-ring cell histology according to WHO (14). As a validation set, 43 prospectively collected endoscopic biopsies and plasma samples from gastric cancer patients with similar characteristics to that of the testing set were obtained together with plasma samples from 31 asymptomatic age- and gender-matched controls. All samples were immediately snap-frozen at the time of surgery or endoscopic procedure. None of cases had family history of gastric cancer. This study was approved by the Comité de Ética of the Central Metropolitan Service and the Medicine School of the Pontificia Universidad Católica de Chile.

DNA Extraction

Samples of Gastric Tissue

Five sections of 15 μm from representative areas of gastric cancer were cut and placed in a 0.5 ml tube for DNA extraction. The DNA extraction was performed in 100 μl of extraction solution [1 mol/l Tris (pH 8.0), 50 mmol/l EDTA, 0.5% and Tween 20] with 1 mg/ml Proteinase K (Sigma) during 12 hours at 55° C. The Proteinase K was inactivated by boiling at 100° C. during 10 minutes, and the DNA was purified by extraction with phenol-chloroform and ethanol precipitation, accordingly to standard protocols. The DNA concentration was determined by spectroscopy employing DO₂₆₀ for 50 μg/ml. The modified DNA was stored at −80° C. until used.

### Plasma Samples

Plasma samples were selected as a source of DNA for analysis based on prior reports indicating that plasma was better for detection of the DNA methylation than the serum (15). For obtaining the plasma, two 4.5 ml tubes (2×4.5 ml) with EDTA additive were collected (lilac-colored stoppers, Vacuette® tubes), permitting separation of the blood by placing the tube in a support at room temperature for 12 hours. Then the supernatant plasma was aseptically separated using a Pasteur pipette. The plasma was transferred to two 1.7 ml tubes, filling a maximum of 1.5 ml in each tube. Both tubes were frozen at −20° C. For DNA extraction from frozen plasma, 200 μl of plasma together with 20 μl of Proteinase K (20 mg/ml) and 200 μl of extraction buffer (AL buffer). The mixture was stirred for 15 seconds, incubated at 56° C. for 10 minutes, and then briefly centrifuged. Then, 200 μl of EtOH (96-100%) were added, it was stirred again and added to the QIAamp column, centrifuged at 6.000 g (8.000 rpm)×1 minute, and once the filtrate was discarded, 500 μl of AW1 buffer was added for performing another centrifugation at 6.000 g (8.000 rpm) for 1 minute. Once discarded the filtrate, 500 μl of AW2 buffer were added, centrifuged at 20.000 g (14.000 rpm) for 3 minutes, and the column was placed into clean 1.5 ml tubes adding 200 μl of AE buffer or distilled water. The mixture was incubated at room temperature (15-25° C.) for 5 minutes followed by centrifugation at 6.000 g (8.000 rpm) for 1 minute for recovering the extracted DNA from the eluted from the column into the clean tube. The DNA concentration was determined by spectroscopy using DO₂₆₀ for 50 μg/ml. The modified DNA was stored at −80° C. until used.

Modification by Sodium Bisulfide

The extracted DNA (gastric tissue and plasma) was treated with sodium bisulfide, according to the technique disclosed previously by the laboratory (16). Briefly, the assay was made taking 1 μg of genomic DNA, denatured by incubation with 0.2 mol/l of NaOH during 10 minutes, at 37° C.; hydroquinone (10 mmol/l, 30 μl; Sigma) was added and 3 mol/l of sodium bisulfide (pH 5.0; 520 μl; Sigma), and the solution was incubated at 50° C. for 16 hours. The treated DNA was purified using the “Wizard DNA purification system” (Promega Corp.), was desulfonated with 0.3 mol/l of NaOH, precipitated with ethanol and resuspended in water. The modified DNA was stored at −80° C. until its amplification by Methylation-Specific Polymerase Chain Reaction.

Methylation-Specific Polymerase Chain Reaction

The Methylation-Specific Polymerase Chain Reaction (MS-PCR), was performed in a volume of 25 μl with 10 mM Tris-HCl, pH 8.0, 50 mM KCl, 1.5 mM Mg Cl, 200 μM dNTP, 0.5 μM starters, 2.5 U Taq polymerase and 4 μl of previously extracted and bisulfide treated DNA. The amplification conditions were initial denaturation at 94° C.×5 minutes×1 time, followed by 40 cycles of denaturation at 94° C.×30 seconds, aligning at 55-63° C.×1 minute and extension at 72° C.×30 seconds; after that a final extension at 72° C.×5 minutes was made. The aligning temperature was experimentally adjusted for each gene included in the study. For each sample, the parallel amplification of the human beta-globine gene was made for controlling the efficiency of the amplification. The result was analyzed by electrophoresis in agarosa gels at 3%, at 200 volts during 20 minutes and was visualized by UV light after dyeing with ethidium bromide at 0.05%. The genes selected by this analysis are shown in Table 1. For each of the genes, triplicate amplifications were performed for the methylated and non methylated conditions.

Reverse Transcription PCR and Post Treatment with Demethylating Agent 5-aza-2′-deoxycytidine

Previously, an association between hypermethylation and gene silencing was established for the 24 selected genes in the assay (16, 33). However, this association between SHP1, APC, Reprimo, FHIT, E-cadherin and SEMA3B was confirmed by reverse transcription-PCR in the cell line MKN45, a cell line of poorly differentiated gastric cancer of feminine mongoloid origin. Total RNA was extracted employing the Qiagen RNeasy System (Qiagen), following the instructions of the manufacturer. The RNA concentration was determined by measuring the absorbency at 260 nm, and the quality was verified by the integrity of the samples 285 and 18S rRNA, after dyeing with ethidium bromide of the total RNA samples that underwent electrophoresis in agarosa gel at 0.8%. The total cDNA was synthesized with the reverse transcriptase from Leukemia Murina Molones virus (ThermoScript RT; Invitrogen). The reverse PCR transcription was made using 1 μg of the total cellular RNA for generating cDNA. The sequences of the starters are already known (29, 31, 32, 34-36). As a control for the RNA charge in the PCR reverse transcription, the glyceraldehydes-3-phosphate dehydrogenase (GAPDH) was used. Ten microliters of each PCR reaction in agarosa gels at 2% were directly charged and separated by electrophoresis. The gels were dyed with ethidium bromide for visualizing them under UV illumination. For restoring the silenced gene expression of the methylated genes, a cell line was treated during 72 hours with the demethylation agent 5-aza-2′-deoxycitidine (Sigma), at a concentration of 2 μmol/l as is already disclosed in the literature (29).

Analysis of the Data

The methylation frequencies were compared with the X² test or Fisher's Exact test. In all tests, the values with a probability P<0.05 were considered statistically significant.

### DESCRIPTION OF THE FIGURES

FIG. 1: PCR Specific methylation analysis of 32 cases of retrospectively collected gastric cancer. A: histogram representing the methylation percentage of the 24 indicated genes. B: Results of the PCR Specific methylation, illustrative for 7 of the studied genes in 6 cases (tracks 1-6). WM: molecular weight marker; M: PCR product with specific starters for the methylated DNA; U: PCR product with specific starters for the non-methylated DNA; CM: Cell line MKN45, methylated in vitro (positive control for methylated genes); CU: DNA from peripheral blood lymphocytes (used as control for non-methylated genes).

FIG. 2: Representative examples of RT-PCR (reverse transcriptase-PCR). Expression of SHP1 and Reprimo in MKN45 cell lines of gastric cancer, before (0 hours) and after (48 hours) of treatment with 5-aza-2′-deoxycitidine (5-azaCdR) are shown. The expression of gene GAPDH was run as RNA integrity control. WM: molecular weight marker; CM: Cell line MKN45, methylated in vitro (positive control for methylated genes); NC: Negative control (water).

FIG. 3: PCR—Specific methylation analysis of 43 cases of gastric cancer retrospectively collected and 31 asymptomatic controls determined by age and sex. A: histogram representing the methylation percentage of the 7 indicated genes. For each gene, 3 columns are made as a graphic representing from left to right samples of the tumor, samples of plasma from individuals with cancer and samples of plasma from asymptomatic control individuals.

B-D: Results of the PCR-specific methylation, illustrative for 2 of the 7 studied genes (APC and Reprimo), in the types tubular/papillary and poorly differentiated (B), muccinuous/cells of the signet seal type (C), and asymptomatic controls (D); WM: molecular weight marker; M: PCR product with specific starters for the methylated DNA; U: PCR product with specific starters for the non-methylated DNA; CM: cell line MKN45, methylated in vitro (positive control for methylated genes); CU: DNA from peripheral blood lymphocytes (used as control for non-methylated genes); T: tumor; P: plasma.

### EXAMPLES

### Results

### Example 1

### Methylation Patterns of the DNA of Island CpG from Multiple Genes

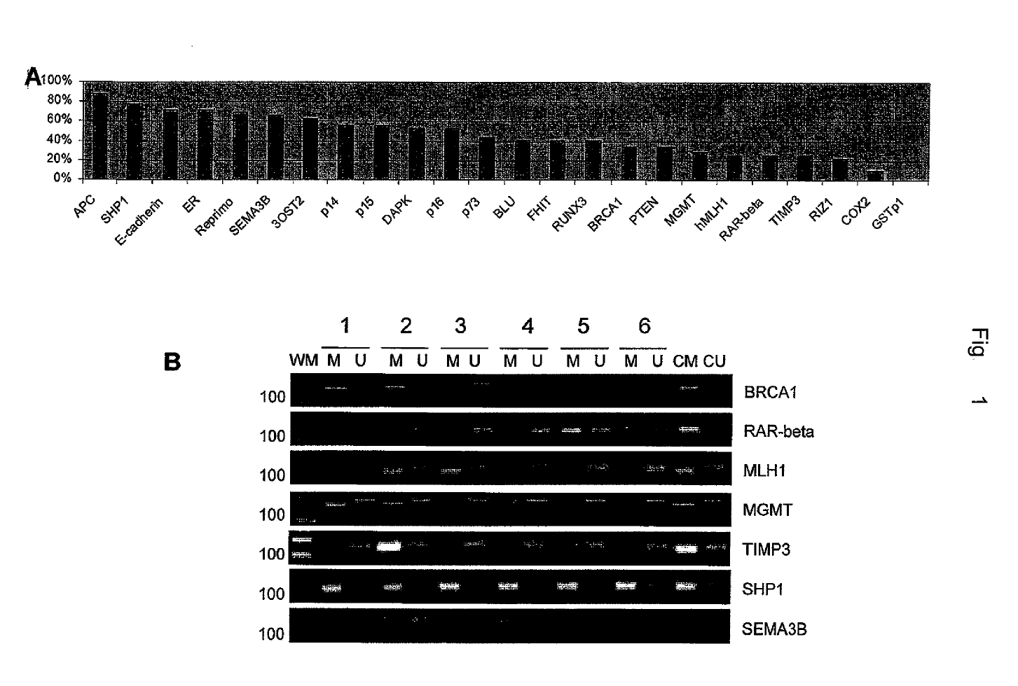

In FIG. 1A, the complete spectrum of the methylation patterns of the DNA of the 24 genes observed in the 32 cases retrospectively collected gastric cancer. Twenty three cases (96%) showed hypermethylation of the promoting region in at least 3 or more of the tested cases, whereas 11 genes were hypermethylated in at least 50% of the cases (APC, SHP1, E-cadherin, ER, Reprimo, SEMA3B, 3OST2, p14, p15, DAPK, and p16). Two of these 11 methylated genes, (SHPT and SEMA3B) had not been reported prior to gastric cancer. Representative examples of the methylation analysis of DNA are shown in FIG. 1B.

### Example 2

### Methylation Patterns of the DNA and Loss of Expression of RNA in Cell Line MKN45

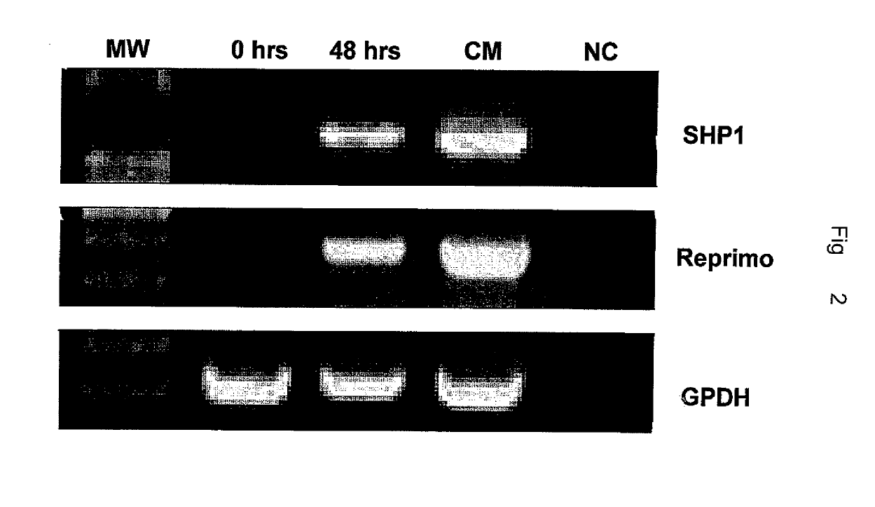

In order to establish the association between the methylation pattern of DNA and the silencing of the gastric cancer genes, the expression of mRNA from 5 of the genes that were methylated in at least 50% of the cases (APC, SHPT, E-cadherin, Reprimo, and SEMA-3B). For this, the cell line MKN45 was treated with the demethylation agent 5-aza-2′-deoxycitidine. As shown in FIG. 2, (genes SHP1 and Reprimo, representatives of the 5 analyzed genes), the reactivation of the gene expression was associated with the addition of the demethylation drug.

### Example 3

### Clinical-Pathological Associations

The relationship between the clinical and pathological relations of the cases and the methylation patterns of the DNA in the group of tested cases was determined. The methylation of 8 genes (BRCA1, p73, RARβ, hMLH1, RIZI, RUNX3, MGMT, and TIMP3) was statistically associated with a gastric cancer variant, cells in the signet ring seal (P=0.03 by Fisher's Exact test). These data suggest that, at the molecular level, the cells in the signet-ring cell are considered a subtype of gastric cancer. None other clinical or pathological association was found, either by analysis of only one gene or multiple genes.

### Example 4

### DNA Methylation Patterns of CpG Island Prospectively Collected in Case of Gastric Cancer and Asymptomatic Controls

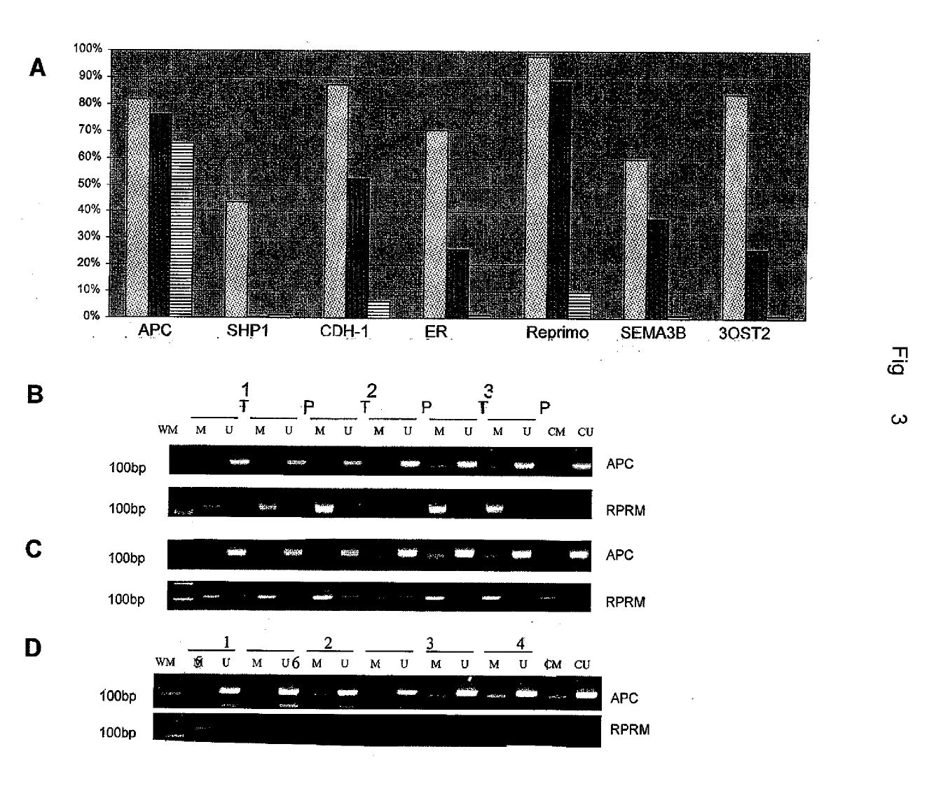

The seven genes with the higher hypermethylation frequency (APC, SHPI, E-cadherin, ER, Reprimo, SEMA3B, and 3OST2) were analyzed in 43 cases of gastric cancer prospectively collected for evaluating the significance of the DNA methylation patterns of the CpG island as clinical biomarkers for the early diagnosis of gastric cancer. In this assay, a biopsy of the gastric tumor and plasma samples were obtained. The same seven genes were studied in 31 plasma samples collected from asymptomatic controls, adjusted for age and sex. FIG. 3A shows that only the Reprimo methylation was identified in the 97.7% (41 of 43) of tumor and plasma from patients with gastric cancer, respectively. However, among the asymptomatic controls, the identification of the Reprimo methylation was only 9.7% (3 of 31) of the proven cases. These differences were statistically significant (p<0.00001 by the Fisher's Exact test). Despite the methylation of the APC, that was frequent in tumor and plasma samples from patients with gastric cancer, differences with the plasma samples of asymptomatic controls were not observed. Representative examples of these analyses are shown in FIG. 3B-D. The other five genes (SHPI, E-cadherin, ER, SEMA3B, and 3OST2) though were frequently methylated in tumor samples, presented a low methylation percentage in their corresponding plasma samples (FIG. 3A).

With the prior antecedents and with the surprising results encountered by the inventors, an early diagnosis method for gastric cancer was developed in blood samples, for asymptomatic individuals.

The objective of this early diagnosis method is to find out those initial gastric cancer cases for a larger control and opportune treatment of the disease, with the corresponding advantage of a better prognosis and quality of life for these individuals.

The diagnosis method essentially consists in determining the Reprimo gene hypermethylation in blood samples, specifically in plasma from individuals that do not show symptoms or signs of gastric cancer.

Example of the Diagnosis Method:

Obtaining Blood Samples from Asymptomatic Individuals

The asymptomatic individuals that underwent this test were healthy, over 40 years old individuals. The focus might be similar to the one for the specific prostatic antigen for prostate cancer. For the study, 10 ml of complete blood are required that are placed in two 4.5 ml tubes (2×4.5 ml) with the EDTA additive (lilac-colored stopper, Vacuette® tubes) and sent to the laboratory in less than 12 hours. The remnant blood not utilized in the determination is discarded.

Obtaining Plasma Samples from the Blood Samples

For obtaining plasma from blood with EDTA additive (lilac-colored stopper, Vacuette® tubes), the tube is placed in a support at room temperature during 12 hours. Then, 1.5 ml (in duplicate) of plasma is suctioned with a Pasteur pipette. The plasma is transferred into 1.7 ml tubes, and both tubes are frozen at −20° C. until DNA extraction.

DNA Extraction from Plasma

For extracting the DNA, 0.2 ml of plasma together with 20 μl of Proteinase K (20 mg/ml) and 200 μl of extraction buffer (AL buffer) are taken. The mixture is shaken for 15 seconds, incubated at 56° C. for 10 minutes, and then briefly centrifuged. Then, 200 μl of EtOH (96-100%) are added, admixed again and added to the QIAamp column. The column is centrifuged at 6.000 g (8.000 rpm)×1 minute, and once the filtrate is discarded, 500 μl of AW1 buffer are added, centrifuged again at 6.000 g (8.000 rpm)×1 minute. Once discarded the filtrate, 500 μl of AW2 buffer are added and centrifuged at 20.000 g (14.000 rpm)×3 minutes and the column is placed into 1.5 ml clean tubes, adding 200 μl of AE buffer and incubating at room temperature (15-25° C.)×5 minutes, followed by centrifuged at 6.000 g (8.000 rpm)×1 minute, obtaining pure DNA in the eluted portion. The DNA concentration on this eluted portion is determined by spectroscopy using DO₂₆₀ for 50 μg/ml. The extracted DNA is stored at −80° C. until its analysis. Previously, the extracted DNA is treated with sodium bisulfite. For it, 1 μg of the extracted DNA is denatured by incubation with 0.2 mol/l of NaOH during 10 minutes at 37° C., adding hydroquinone (10 mmol/l, 30 μl; Sigma) and 3 mol/l of sodium bisulfide (pH 5.0; 520 μl; Sigma). The solution is incubated at 50° C. during 16 hours. Later on, the DNA is purified by means of columns (“Wizard DNA purification system”, Promega Corp.), and desulfonated with 0.3 mol/l of NaOH, precipitated with ethanol and resuspended in water. The modified DNA is stored at −80° C. until its amplification by Specific Methylation-Polymerase Chain Reaction.

Specific Methylation-Polymerase Chain Reaction

For determining the presence of methylated Reprimo, the Specific Methylation-Polymerase Chain Reaction (MS-PCR) is performed.

For amplification of the methylated sequence of Reprimo, the starters used are:

GCGAGTGAGCGTTTAGTTC/TACCTAAACCGAATTCATCG.

For the amplification of the Non Methylated sequence of Reprimo, the used starters are:

TTGTGAGTGAGTGTTTAGTTTG/TAATTACCTAAAACCAAATTCATC

The size of the amplified product is 112 pair of bases. The amplifying conditions were previously disclosed, with the only modification in the starter's temperature (57° C.).

For the PCR reaction, in a volume of 25 μl, 10 mM of Tris-HCl (pH 8.0, 50 mM KCl, 1.5 mM MgCl, 200 μM dNTP, 0.5 μM starters, 2.5 U Taq polymerase, and 4 μl of the extracted and bisulfide modified DNA. The amplification conditions are initial denaturing at 94° C.×5 minutes×1 time, followed by 40 denaturing cycles at 94° C.×30 seconds, aligning at 55-63° C.×1 minute and extension at 72° C.×30 seconds; later a final extension at 72° C.×5 minutes will follow. For each analyzed sample, a parallel amplification of the Myo-D gene is performed for controlling the amplification efficiency, and in particular, the validity of the negative result. The result is analyzed by electrophoresis in agarosa gels at 3%, at 200 volts during 20 minutes and visualization by UV light after dyeing with ethidium bromide at 0.05%. For each sample analyzed, triplicate amplifications were performed.

Interpretation of the Positive Result for Amplification of Methylated Reprimo and Gastric Cancer

In the case the detection results positive for the methylated Reprimo in an asymptomatic individual (probability of 1 in 300 individuals), the positive individuals for Reprimo will undergo an endoscopic procedure, with biopsy extraction and histological analysis in case of encountering some histological alteration. The obtained results demonstrate that the presence of Reprimo in the plasma is in more than 90% of the patients with gastric cancer, and only in 10% of the healthy individuals. These results endorse the possibility of using this diagnostic method for gastric cancer in preventive form, because there exists a direct correlation between the Reprimo gene into the plasma and the higher probability of gastric cancer appearance.

On the other hand, the results obtained with the diagnostic method permit establishing health policies in such a form that those individuals that resulted positive by this diagnostic should undergo other diagnostic tests and be controlled continuously and periodically for treating the gastric cancer at the moment of its appearance, implying a higher probability of survival in relation to an individual without early diagnostic.

### Discussion

Numerous attempts had been made for defining the methylation patterns of the DNA for each human cancer type (16, 21, 37-42). The first documented epigenetic alteration in gastric cancer was the hypermethylation of the DNA repairing genes (hMSH2 and hMLH1, references 8, 43). Though several epigenetically inactivated genes had been disclosed (5, 9-11, 18, 44-46), an understandable methylation profile for gastric cancer had not been achieved yet. In this study, 24 genes were used, from which 8 (BRCAI, p73, RAR-beta, hMLH1, RIZI, RUNX3, MGMT, and TIMP3) were associated with an aggressive variant of gastric cancer, the signet-ring cell cancer. Recent studies suggested that signet-ring cell are epidemiologic, clinically-pathologically and molecularly a different subtype of gastric cancer (47, 48). Therefore, the findings of the invention not only identify the methylation profile of these variant of gastric cancer, but also supports the hypothesis that hypermethylation of CpG islands does not occur at random, but through a process of specific selection of key tumor suppressor genes (41). Present study also identified other significantly methylated genes of gastric cancer (SHPI and SEMA3B). The SHPI gene (tyrosynfosfatase specific for Hematopoietic cellular protein) is a member of the pathway JAK-STAT and is located in 12p13. Frequently, this gene had been described as inactivated by methylation in the leukemia and the lymphomas (49), and more recently, in the gall bladder carcinoma (21). The SEMA3B gene, a member of the tumoral suppressor cluster 3p21.3 had been described as inactivated by methylation in several tumors such as the liver, the gall bladder, lung and ovary (16). This is the first report on methylation of SHPI and SEMA3B in gastric cancer.

Several studies deal with the usefulness of the epigenetic biomarkers for human cancer detection (7). Methylation anomalies had been detected is blood, or sputum from patients with lung cancer, in serum or plasma samples of head and neck cancer patients, duct washing fluid from patients with breast cancer, and in the urine of patients with prostate and bladder cancers (7). In order to explore the usefulness of the diagnostic with epigenetic biomarkers in gastric cancer detection, the genes most frequently hypermethylated (APC, SHPI, E-cadherin, ER, Reprimo, SEMA3B, and 3OST2), in retrospective samples were evaluated. In this validation, the high frequency of methylation in primary tissues from all the evaluated genes was confirmed. However, only two genes (APC and Reprimo) were frequently methylated (>70%) in the corresponding plasma samples, as disclosed in Example 4 and in FIG. 3. When these genes were evaluated among the asymptomatic controls plasma samples, only the Reprimo gene was significantly less methylated than the others. The obtained results by present invention agree with former results, wherein frequently the methylated Reprimo gene was encountered in several cancer types, but seldom in non-malignant tissues (29). However, the plasma results obtained by present invention are the first studies that permit indicating that the Reprimo gene is useful as biomarker for the early detection of gastric cancer.

The results from the disclosed examples permit providing an early and efficient diagnostic method for gastric cancer, which is reinforced by prior studies wherein Reprimo is a mediator after the expression of p53 induced by the arrest of G2 from the cell cycle (50). When Reprimo is over-expressed, the arrest of the cell cycle in phase G2 is induced, suggesting that it has a tumor suppression function (50). Because of the functional suppression of the tumor suppressor p53, the genes and their mediators such as 14-3-3-δ-, Reprimo, are fundamental for the development of human cancers.

The inventors published preliminary results from present invention in the publication “Reprimo as a potential biomarker for early detection in gastric cancer” (55).

### REFERENCES

1. Parkin D M. International variation. Oncogene 2004; 23:6329-40.
2. Rivera F, Vega-Viilegas M E, Lopez-Brea M F. Chemotherapy of advanced gastric cancer. Cancer Treat Rev 2007; 33:315-24.
3. Nomura S, Kaminishi M. Surgical treatment of early gastric cancer. Dig Surg 2007; 24:96-100.
4. Yasui W, Oue N, Aung P P Matsumura S, Shutoh M, Nakayama H. Molecular-pathological prognostic factors of gastric cancer: a review. Gastric Cancer 2005; 8:86-94.
5. Tamura G. Alterations of tumor suppressor and tumor-related genes in the development and progression of gastric cancer. World J. Gastroenterol. 2006; 12:192-8.
6. Callinan P A, Feinberg A P. The emerging science of epigenomics. Hum Mol Genet 2006; 15 Suppl 1: R95-101.
7. Ushijima T. Detection and interpretation of altered methylation patterns in cancer cells. Nat Rev Cancer 2005; 5:223-31.
8. Fleisher A S, Esteller M, Wang S, et al. Hypermethylation of the hMLH1 gene promoter in human gastric cancers with microsatellite instability. Cancer Res 1999; 59:1090-5.
9. Kang G H, Lee S, Kim J S, Jung H Y. Profile of aberrant CpG island methylation along the multistep pathway of gastric carcinogenesis. Lab Invest 2003; 83:635-41.
10. Kim H C, Kim J C, Roh S A, et al. Aberrant CpG island methylation in early-onset sporadic gastric carcinoma. J Cancer Res Clin Oncol 2005; 131:733-40.
11. Oue N, Mitani Y, Motoshita J, et al. Accumulation of DNA methylation is associated with tumor stage in gastric cancer. Cancer 2006; 106:1250-9.
12. Retamales E, Rodriguez L, Guzman L, et al. Analytical detection of immunoglobulin heavy chain gene rearrangements in gastric lymphoid infiltrates by peak area analysis of the melting curve in the LightCycler System. J Mol Diagn 2007; 9:351-7.
13. Fenoglio-Preiser C. Carneiro F, Correa P. et al. Gastric Carcinoma. In: Hamilton H B, Aaltonen L, editors. Pathology and Genetics of Tumours of the Digestive System. Lyon: IARC Press; 2000. p. 37-68.
14. Fenoglio-Preiser C, Carneiro F, Correa P. et al. Gastric Carcinoma. In: Hamilton H B, Aaltonen L, editors. Pathology and Genetics of Tumours of the Digestive System. Lyon: IARC Press; 2000 p37-68.
15. Jung M, Klotzek S, Lewandowski M, Fleischhacker M, Jung K. Changes in concentration of DNA in serum and plasma during storage of blood samples. Clin Chem 2003: 49:1028-9.
16. Riquelme E, Tang M, Baez S, et al. Frequent epigenetic inactivation of chromosome 3p candidate tumor suppressor genes in gallbladder carcinoma. Cancer Lett 2007; 250:100-6.
17. Miyamoto K, Asada K, Fukutomi T. et al, Methylation-associated silencing of heparan sulfate D-glucosaminyl 3-O-sulfotransferase-2 (3-OST-2) in human breast, colon, lung and pancreatic cancers. Oncogene 2003; 22:274-80.
18. Sarbia M, Geddert H, Klump B, Kiel S, Iskender E, Gabbert H E. Hypermethylation of tumor suppressor genes (p161NK4A, p14ARF and APC) in adenocarcinomas of the upper gastrointestinal tract. Int J Cancer 2004:111:224-8.
19. Chan K Y, Ozcelik H, Cheung A N, Ngan H Y, Khoo U S. Epigenetic factors controlling the BRCA1 and BRCA2 genes in sporadic ovarian cancer. Cancer Res 2002; 62:4151-6.
20. Xu X L, Yu J, Zhang H Y. et al. Methylation profile of the promoter CpG islands of 31 genes that may con tribute to colorectal carcinogenesis. World J Gastroenterol 2004: 10:3441-54.
21. Takahashi T, Shivapurkar N, Riquelme E, et al. Aberrant promoter hypermethylation of multiple genes in gallbladder carcinoma and chronic cholecystitis. Clin Cancer Res 2004; 10:6126-33.
22. Mori T, Martinez S R, O'Day S J, et al. Estrogen receptor −α methylation predicts melanoma progression. Cancer Res 2006; 66:6692-8.
23. Kim J, Lee H S, Bae S I, Lee Y M, Kim W H. Silencing and CpG island methylation of GSTP1 is rare in ordinary gastric carcinomas but common in Epstein-Barr virus-associated gastric carcinomas. Anticancer Res 2005; 25:4013-9.
24. Kawaguchi K, Oda Y, Saito T. et al. DNA hypermethylation status of multiple genes in soft tissue sarcomas. Mod Pathol 2006; 19:106-14.
25. Kim H C, Roh S A, Ga I H, Kim J S, Yu C S, Kim J C. CpG island methylation as an early event during adenoma progression in carcinogenesis of sporadic colorectal cancer. J Gastroenterol Hepatol 2005; 20:1920-6.
26. Liu Z, Wang L E, Wang L, et al. Methylation and messenger RNA expression of p15INK4b but not p16INK4a are independent risk factors for ovarian cancer. Clin Cancer Res 2005; 11:4968-76.
27. Sato K. Tamura G, TsuchiyaT et al. Analysis of genetic and epigenetic alterations of the PTEN gene in gastric cancer. Virchows Arch 2002; 440:160-5.
28. Virmani A K, Rathi A, Zochbauer-Muller S, et al. Promoter methylation and silencing of the retinoic acid receptor-β gene in lung carcinomas. J Natl Cancer Inst 2000; 92:1303-7.
29. Takahashi T, Suzuki M, Shigematsu H, et al. Aberrant methylation of Reprimo in human malignancies. Int J Cancer 2005; 115:503-10.
30. Li Q L, Ito K, Sakakura C, et al. Causal relationship between the loss of RUNX3 expression and gastric cancer. Cell 2002; 109:113-24.
31. Kuroki T, Trapasso F, Yendamuri S, et al. Allelic loss on chromosome 3p21.3 and promoter hypermethylation of semaphorin 3B in non-small cell lung cancer. Cancer Res 2003; 63:3352-5.
32. Oka T, Ouchida M, Koyama M, at al. Gene silencing of the tyrosine phosphatase SHP1 gene by aberrant methylation in leukemias/lymphomas. Cancer Res 2002; 62:6390-4.
33. Wild A, Ramaswamy A, Langer P. et al. Frequent methylation-associated silencing of the tissue inhibitor of metalloproteinase-3 gene in pancreatic endocrine tumors. J Clin Endocrinol Metab 2003; 88:1367-73
34. Deng G, Song G A, Pong E, Sleisenger M, Kim Y S. Promoter methylation inhibits APC gene expression by causing changes in chromatin conformation and interfering with the binding of transcription factor CCAAT-binding factor. Cancer Res 2004; 64:2692-8.
35. Iwai M, Kiyoi H, Ozeki K, et al. Expression and methylation status of the FHITgene in acute myeloid leukemia and myelodysplastic syndrome. Leukemia 2005; 19:1367-75.
36. Takeno S, Noguchi T, Fumoto S, Kimura Y, Shibata T, Kawahara K. E-cadherin expression in patients with esophageal squamous cell carcinoma: promoter hypermethylation, Snail overexpression, and clinicopathologic implications. Am J Clin Pathol 2004; 122:78-84.
37. Shames D S, Girard L, Gao B, et al. A genome-wide screen for promoter methylation in lung cancer identifies novel methylation markers for multiple malignancies. PLoS Med 2006; 3:e486.
38. Shames D S, Minna J D, Gazdar A F. Methods for detecting DNA methylation in tumors: From bench to bedside. Cancer Lett 2007; 251:187-98.
39. Plass C, Smiraglia D J. Genome-wide analysis of DNA methylation changes in human malignancies. Curr Top Microbiol Immunol 2006; 310:179-98.
40. Esteller M. Cancer epigenetics: DNA methylation and chromatin alterations in human cancer. Adv Exp Med Biol 2003; 532:39-49.
41. Parrella P, Poeta M L, Gallo A P at al. Nonrandom distribution of aberrant promoter methylation of cancer related genes in sporadic breast tumors. Clin Cancer Res 2004; 10:5349-54.
42. Alaminos M, Davalos V, Cheung N-K V, Gerald W L, Esteller M. Clustering of gene hypermethylation associated with clinical risk groups in neuroblastoma. J Natl Cancer Inst 2004; 96:1208-19.
43. Fleisher A S, Estelrer M, Tamura G, et al. Hypermethylation of the hMLH1 gene promoter is associated with microsatellite instability in early human gastric neoplasia. Oncogene 2001; 20:329-35.
44. Leung W K, Yu J, Ng E K, et al. Concurrent hypermethylation of multiple tumor-related genes in gastric carcinoma and adjacent normal tissues. Cancer 2001; 91: 2294-301.
45. Tamura G, Yin J, Wang S, at al. E-Cadherin gene promoter hypermethylation in primary human gastric carcinomas. J Natl Cancer Inst 2000; 92:569-73.
46, To K F, Leung W K, Lee T L, et al. Promoter hypermethylation of tumor-related genes in gastric intestinal metaplasia of patients with and without gastric cancer. Int J Cancer 2002; 102:623-8.
47, Kim D Y, Park Y K, Joo J K, et al. Clinicopathological characteristics of signet ring cell carcinoma of the stomach. ANZ J Surg 2004; 74:1060-4.
48. Chang Y T, Wu M S, Chang C J, Huang P H, Hsu S M, Lin J T. Preferential loss of Fhit expression in signet ring cell and Krukenberg subtypes of gastric cancer. Lab Invest 2002; 82:1201-8.
49. Hayslip J, Montero A. Tumor suppressor gene methylation in follicular lymphoma: a comprehensive review. Mol Cancer 2006; 5:44.
50. Ohki R, Nemoto J, Murasawa H, at al. Reprimo, a new candidate mediator of the p53-mediated cell cycle arrest at the G2 phase. J Biol Chem 2000; 275:22627-30.
51. Leung W K, To K F, Chu E S, et al. Potential diagnostic and prognostic values of detecting promoter hypermethylation in the serum of patients with gastric cancer. Br J Cancer 2005; 92:2190-4.
52. Lee T L, Leung W K, Chan M W, et al. Detection of gene promoter hypermethylation in the tumor and serum of patients with gastric carcinoma. Clin Cancer Res 2002; 8:1761-6.
53. Miyamoto A, Kuriyama S, Nishino Y, et al. Lower risk of death from gastric cancer among participants of gastric cancer screening in Japan: a population-based cohort study. Prey Med 2007; 44:12-9.
54. Rollan A, Ferreccio C, Gederlini A, Serrano C, Torres J, Harris P. Non-invasive diagnosis of gastric mucosal atrophy in an asymptomatic population with high prevalence of gastric cancer. World J Gastroenterol 2006; 12:7172-8.
55. Bernal C, Aguayo F, Villarroel C, Vargas M, Diaz I, Ossandon F, Santibáñez E, Palma M, Aravena E, Barrientos C, and Corvalan A H. Reprimo as a potential biomarker for early detection in gastric cancer. Clin Cancer Res 2008; 14 (19) 6264-9.

What is claimed is:

1. A detection method for early detection of gastric cancer, CHARACTERIZED in that it is a non-invasive method comprising the stages of:
a) obtaining human plasma samples;
b) detecting the presence of specific markers for gastric cancer in plasma samples of stage a).

2. The detection method from claim 1, CHARACTERIZED in that the gastric cancer markers detected in the plasma correspond to DNA molecules.

3. The detection method from claim 2, CHARACTERIZED in that the plasmatic DNA molecule corresponds to a methylated DNA molecule.

4. The detection method from claim 3, CHARACTERIZED in that the DNA molecule methylations corresponds to aberrant methylations.

5. The detection method from claim 3, CHARACTERIZED in that the methylated DNA molecule is a part of the Reprimo gene.

6. The detection method from claim 5, CHARACTERIZED in that the methylated DNA molecule corresponds to the promoter for the Reprimo gene.

7. The detection method from claim 2, CHARACTERIZED in that the DNA molecule obtained from plasma is determined by means of the Specific Methylation-Polymerase Chain Reaction.

8. The method from claim 7, CHARACTERIZED in that the Specific Methylation-Polymerase Chain Reaction is performed using the following starters:
GCGAGTGAGCGTTTGTTC
TACCTAAAACCGAATTCATCG.

9. The early detection method for gastric cancer from claim 1, CHARACTERIZED in that it is useful for application in gastric cancer massive detection programs.

10. The early detection method for gastric cancer from claim 1, CHARACTERIZED in that it is useful for application in gastric cancer prevention programs.

11. The method from claim 1, CHARACTERIZED in that it comprises the following stages:
a) obtaining human plasma samples;
b) isolation of the plasma DNA;
c) Amplification of the specific DNA by means of the Specific Methylation-Polymerase Chain Reaction for the Reprimo gene.
d) determination of the presence or absence of the methylated Reprimo gene in the plasma.


SEQUENCE LISTING (4 sequences)

SEQ ID NO: 1 - DNA, 19 bp, Artificial Sequence
  primer for amplification of methylated sequence of Reprimo
gcgagtgagcgtttagttc

SEQ ID NO: 2 - DNA, 20 bp, Artificial Sequence
  primer for amplification of methylated sequence of Reprimo
tacctaaaccgaattcatcg

SEQ ID NO: 3 - DNA, 22 bp, Artificial Sequence
  primer for amplification of non-methylated sequence of Reprimo
ttgtgagtgagtgtttagtttg

SEQ ID NO: 4 - DNA, 24 bp, Artificial Sequence
  primer for amplification of non-methylated sequence of Reprimo
taattacctaaaaccaaattcatc